(19) United States
(12) Patent Application Publication
(10) Pub. No.: US 2012/0065079 A1
(43) Pub. Date: Mar. 15, 2012

(54) METHOD FOR THE DETERMINATION OF P BLOOD GROUPS

(76) Inventors: Martin L. Olsson, Lund (SE); Britt Thuresson, Kavlinge (SE)

(21) Appl. No.: 13/318,683

(22) PCT Filed: May 7, 2010
(86) PCT No.: PCT/SE10/50504

§ 371 Date: Nov. 3, 2011

(30) Foreign Application Priority Data
May 7, 2009 (SE) 0900629-7

(51) Int. Cl. C12Q 1/68; C07H 21/04; C40B 20/00

(52) U.S. Cl. 506/2; 435/6.11; 536/24.31; 536/24.33

(57) ABSTRACT

The invention relates to a method to discriminate between the P¹ and P² blood group alleles by the use of at least one nucleotide sequence being identical/homologous to at least part of the nucleotide sequences as shown in SEQ ID NO:1-6 or a nucleotide sequence showing at least 90% identity to any of SEQ ID NO:1-6 and wherein the difference is a cytosine (C) or thymidine (T) in position 129 as shown in SEQ ID NO:1, wherein C/C or C/T in the two alleles gives rise to the P1 phenotype and T/T to the P₂ phenotype. Furthermore, zygosity for the P¹ allele (i.e. C/C vs. C/T) also predicts the P1 and P^{k} antigen levels on man cells. The invention also relates to means to be used in said method including primers, probes and markers.

### FIELD OF INVENTION

The invention relates to a method to discriminate between the P¹ and P² blood group alleles by the use of at least one nucleotide sequence being identical/homologous to at least part of the nucleotide sequences as shown in SEQ ID NO: 1-6 or a nucleotide sequence showing at least 90% identity to any of SEQ ID NO:1-6 and wherein the difference is a cytosine (C) or thymidine (T) in position 129 as shown in SEQ ID NO:1, wherein C/C or C/T in the two alleles gives rise to the P₁ phenotype and T/T to the P₂ phenotype. Furthermore, zygosity for the P¹ allele (i.e. C/C vs. C/T) also predicts the P1 and P^{k} antigen levels on human cells. The invention also relates to means to be used in said method including primers, probes and markers.

### BACKGROUND OF INVENTION

The genetic basis is now known for 29 out of 30 histo-blood group systems currently recognized by the International Society of Blood Transfusion. The only remaining system without a defined genetic locus is the P blood group system. This system was discovered already in 1927 and encodes the P1 antigen that was determined during the 1960s to be of carbohydrate nature and later more specifically an alpha1-4Galactose coupled to a paragloboseries precursor oligosaccharide. Thus, a galactosyltransferase is the enzyme responsible for synthesis of the P1 antigen. For other blood groups including other carbohydrate ones like ABO, the genetic basis has been utilised to make DNA-based typing possible.

Blood group phenotypes are presently determined using commercially available government-regulated serological reagents and human red cells. These known tests rely on the principle of antibody binding and red cell agglutination to identify clinically relevant blood group phenotypes. The presently known tests were originally devised many years ago and today require the use of government regulated and approved serological reagents. For example, regulatory bodies or documents like the Food and Drug Administration (FDA) in the USA or the In Vitro Diagnostica Directive (IVDD) in the EU provide rules and requirements for use in clinical practice. Some of the tests being employed today have been automated (for example, ABO and Rh typing) or semi-automated. However, still many of the presently used tests are performed manually by highly-trained laboratory technologists and are done on a test-by-test basis. In other words, a technologist must perform four separate tests to determine, for example, the presence or absence of Fy^{a}, Fy^{b}, Jk^{a} and Jk^{b} antigens to phenotype a single blood unit following donation. Essentially, the current tests which employ government-approved reagents in a manual, single-test driven method are a very cost-ineffective method for a blood collection facility that is often required to perform such tests on a high volume basis. Genomic typing has now surfaced as a possible way to automate typing for all blood groups for which the molecular genetic basis is known.

The prior art uses two basic techniques to detect SNPs; polymerase chain reaction-restriction fragment length polymorphism (PCR-RFLP), and allele- or sequence-specific primer (ASP or SSP)-PCR. For PCR-RFLP analysis, restriction enzymes are used to digest PCR-amplified genomic DNA fragments. In brief, DNA is extracted from nucleated blood cells for each blood sample to be analyzed. The PCR is set up manually; a separate PCR is often performed on each sample for each SNP of interest. The PCR-amplified fragments are digested with a specific restriction enzyme and the cleaved products are separated on a gel. The pattern of digested DNA fragments viewed from the gel predicts the presence or absence of either nucleotide of a SNP of interest. In ASP/SSP-PCR, two PCRs are often set up in separate tubes for each SNP of interest. One tube contains a universal primer and a primer with a sequence that is specific to detect one nucleotide of a SNP. The other tube often contains the same universal primer and a primer specific for the other nucleotide of a SNP. Prior art has used two pair or three primer pair PCR to analyze a nucleotide for a given SNP, with at least one pair acting as an internal control to ensure DNA and other critical reagents are available for PCR amplification. Multiplex PCR can also be set up to determine several SNPs simultaneously. Although several alternative PCR-related methods including realtime-PCR, pyrosequencing, allelic discrimination and others have also been used, the prior art does not employ methods to detect blood group SNPs in an automated high-throughput fashion.

The latest technology for this purpose using microarray platforms and is currently being installed at progressive blood centres, mainly in Europe and the USA initially. The underlying idea is to be able to match better the blood compatibility between the donor and recipient of blood and potentially also transplanted organs. Thus, the volumes (=business opportunities) here are quite remarkable. In Sweden alone, almost half a million transfusions are given each year. The ultimate goal is to increase patient safety by decreasing the frequency of serious adverse reactions to blood transfusion, the majority of which including fatalities are due to blood group problems according to data from European and American haemovigilance systems.

Whilst the chromosomal location of P1 gene has been known for years to be the long arm of chromosome 22, the polymorphism giving rise to P1 positive (also called the P₁ phenotype) or P1 negative (also called the P₂ phenotype) red blood cells and tissues is not known. A paper in 2003 [Iwamura K, Furukawa K, Uchikawa M, Sojka B N, Kojima Y, Wiels J, Shiku H, Urano T, Furukawa K]. The blood group P1 synthase gene is identical to the Gb3/CD77 synthase gene. A clue to the solution of the P1/P2/p puzzle. J Biol Chem. 2003; 278:44429-38] claimed to have found such a genetic marker among a limited group of Japanese samples in the regulatory (5′) end of the A4GALT gene, a glycosyltransferase gene known since 2000 [Steffensen R, Carlier K, Wiels J, Levery S B, Stroud M, Cedergren B, Nilsson Sojka B, Bennett E P, Jersild C, Clausen H. Cloning and expression of the histo-blood group Pk UDP-galactose: Galbeta-4Glcbeta1-cer alpha1, 4-galactosyltransferase. Molecular genetic basis of the p phenotype. J Biol Chem. 2000 ;275:16723-9] to encode the enzyme synthesizing another carbohydrate blood group called Pk, CD77 or Gb3. However, two research groups disputed those findings in 2005 [Hellberg A, Chester Mass., Olsson ML. Two previously proposed P1/P2-differentiating and nine novel polymorphisms at the A4GALT (P^{k}) locus do not correlate with the presence of the P1 blood group antigen. BMC Genet. 2005; 6:49 (11 pages electronically published, doi:10.1186/1471-2156-6-49)] and 2006 [Tilley L, Green C, Daniels G. Sequence variation in the 5′ untranslated region of the human A4GALT gene is associated with, but does not define, the P1 blood-group polymorphism. Vox Sang. 2006; 90(3):198-203], respectively, because the implicated two SNPs did not match fully with the P1/P2 phenotypes in the larger sample cohorts tested by these investigators. Thus, genetic testing for the P¹/P² alleles is not possible due to lack of a marker with appropriate concordance to phenotype. Therefore there is a need for a marker to be able to discriminate between the P₁/P₂ phenotypes so that this test can eventually be added to the arsenal of blood group SNPs in future automation efforts. This has potential implications for both transfusion and transplantation medicine as well as susceptibility testing in infectious medicine since the P1 antigen and its relatives P and Pk are used as host cell receptors by a range of bacterial, viral and parasite pathogens causing a variety of diseases.

### SUMMARY OF THE INVENTION

The present invention provides a method of detecting the presence or absence of nucleotides relating to the P¹/P² genotypes as well as the expression of the P1 antigen and/or the PK expression. The inventors have found a new exon (herein termed exon 2a) in the A4GALT gene intron 1 which has been determined to contain a polymorphic site that discriminates between the P₁ and P₂ blood group phenotypes. This sequence can be found in Genbank, reference assembly NC_{—}000022.9, wherein the previously known exon 1 is represented by nucleotides 9-74 and the novel exon 2a is 2285-2625. This new transcript has been found in erythroid cells cultured from CD34+human bone marrow cells and defined by 5′-RACE and 3′-RACE to consist of the above-mentioned nucleotides, possess a 5′ cap and a 3′ polyadenylation signal resulting in a poly-A tail as should be the case for a true mRNA. Its presence has also been confirmed in immortalized human cell lines.

The P¹/P² polymorphism can be found at position 2326 in NC000022 and is therefore part of the new transcript. It can be used to predict the P₁/P₂ phenotypes following genetic analysis. Homozygosity for C (P¹) at position 2326 results in high P1 antigen expression whilst heterozygosity for C (P¹) and T (P²) results in low P1 expression. Homozygosity for T at position 2326 results in the P1-negative P₂ phenotype. Similarly, the P^{k} blood group antigen levels can be predicted by analysis of the same nucleotide position and follows the same pattern as described above for P1. Position 2406 can be either a G or a T in the P² allele but has so far only been found to be T in the P′ allele. One P² variant has an additional deletion in exon 1, i.e., nucleotides 35-59. These variations do not affect P₁/P₂ status per se.

The invention also relates to a kit comprising a set of oligonucleotide primers being homologous to the nucleotide sequence shown in SEQ ID NO:1-6 and wherein said set of oligonucleotide primers is suitable for amplifying and/or detecting the P¹/P² genotype.

### BRIEF DESCRIPTION OF THE DRAWINGS

FIG. 1 shows the results obtained from EXAMPLE 2 and 3, see below.

FIG. 2 shows the A4GALT gene and two transcript variants.

A. Schematic representation of the A4GALT gene at the top with the three previously known exons in black and a novel exon (here designated exon 2a) in grey. Introns are shown as a white line between exons. The GenBank accession no. NC_{—}00022.10 sequence was used to calculate exon and intron sizes indicated below or above their respective symbols. The traditional (exons 1+2+3) and new (exons 1+2a) transcript is shown with exons 1, 2 and 3 in white and exon 2a in grey.

B. The P1 and P2 variants of the new transcript are shown. The polymorphism specific for P¹/P² alleles (ACG vs. ATG where the SNP is highlighted in bold) and two unspecific polymorphisms (indicated by asterisks) included in the transcripts are shown. In the P² allele, the specific polymorphism gives rise to a potential open reading frame (ORF, hatched) and its start (ATG) and stop (TGA) codons are indicated.

FIG. 3 shows the results obtained with three different antisera against P1 antigen on red blood cells tested with hemagglutination. Five samples each with the three genotypes P¹P¹(▴), P²P²(Δ) and P¹P² () were tested with two monoclonal anti-P1 reagents [Immucore (MAb1), Seraclone (MAb2)] and a polyclonal (PAb) anti-P1 goat antiserum as indicated on the x axis. Agglutination reactions were visually graded between 0 and 4+and registered on the y axis.

FIG. 4 shows the P^{k} and P1 antigen expression on red blood cells measured by FACS analysis. P1 antigen expression shown on the left with a representative histogram and below the collected Mean Fluoroscence Intensity (MFI) values in a bar graph. P^{k} antigen expression is shown to the right. In both cases, the genotypes of the tested cells are indicated below the x axis. As indicated above the histograms the P¹P¹ sample is shown with a dark bold line, the heterozygous P¹P² sample with a grey solid line and the P²P² sample is shown by a dotted line. The P1 and Pk negative pp phenotype sample (negative control) is shown as a filled grey peak and for the P^{k} expression a P₁^{k} sample with massive amounts of P^{k} antigen (positive control) is included and shown with a filled black peak. Independent 2-sample t-test assuming equal variance and 2-tailed distribution was used to determine the significance. Each of the genotype groups (P¹P¹, P¹P² or P²P²) was compared to each other using the XLSTAT 2009 (Addinsoft, N.Y., USA) data analyzer. Data were considered statistically significant with respect to the following criteria (*P value<0.05, **P<0.01, ***P<0.001).

### DETAILED DESCRIPTION OF THE INVENTION

In the context of the present application and invention the following definitions apply:

The term “nucleotide sequence” is intended to mean a sequence of two or more nucleotides. The nucleotides may be of genomic DNA, cDNA, RNA, semisynthetic or synthetic origin or a mixture thereof. The term includes single and double stranded forms of DNA or RNA.

The term “homology” is intended to mean the overall homology of the nucleotide sequence as shown in SEQ ID NO:1-6.

The term “identity” is intended to mean exact identity in the same position of the nucleotide sequence as shown in SEQ ID NO:1-6.

### DESCRIPTION

The inventors have found a new exon in the A4GALT gene intron 1, which has been determined to contain a polymorphic site that discriminates between having a P₁ or P₂ blood group phenotype. Thereby it has for the first time been possible to discriminate genomically between the P₁/P₂ phenotypes and making it possible to set up easy and clinically useful genotyping assays such as the methods mentioned below.

The invention relates to a method for DNA-based blood group genotyping for the phenotypes P₁/P₂. It has been found that a person having C/C or C/T (homozygous or heterozygous, respectively) at the allelic position defined in this application will have the P₁ phenotype, wherein the C and T is present in position 129 as shown in SEQ ID NO 1-3. If the person is homozygous and have T/T in this position, the phenotype will be P₂. A method to discriminate between the P¹ and P² alleles by the use of at least one nucleotide sequence being homologous or complementary to part of the nucleotide sequences as shown in SEQ ID NO: 1-6 or a nucleotide sequence showing at least 90% identity to any of SEQ ID NO:1-6 and wherein the difference between the alleles is a C or T in position 129 as shown in SEQ ID NO:1, wherein a person who is homozygous for the allele with C, or heterozygous for the alleles with C and T has the P₁ phenotype and a person homozygous for T has the P₂ phenotype. Furthermore, homozygosity for C (P¹) results in high P1 antigen expression whilst heterozygosity for C (P¹) and T (P²) results in low P1 expression. The same is also true for the P^{k} blood group antigen.

A method to discriminate between the P¹ and P² alleles by the use of at least one nucleotide sequence being homologous to part of the nucleotide sequences as shown in SEQ ID NO: 1-6 or a nucleotide sequence showing at least 90% identity to any of SEQ ID NO:1-6 and wherein the difference is a C or T in position 129 as shown in SEQ ID NO:1-3, where C/C or C/T give rise to the P¹ phenotype and T/T to the P² phenotype. SEQ ID NO:1-3 shows the new exon 2a in the A4GALT gene intron 1. The sequence shown in SEQ ID NO:1 shows the new exon in a P¹ allele and surrounding nucleotide sequences. SEQ ID NO 2-3 shows the exon in P² alleles with surrounding nucleotide sequences. Additionally there are at least two other polymorphic sites in the nucleotide sequences. SEQ ID NO 4-6 shows the nucleotide sequence of the exon 2a and transcript variants.

The detection of the P₁/P₂ phenotypes may be combined with the detection of other phenotypes and used in technologies such as ultra high-throughput multiplex PCR, designed to detect specific SNPs that represent clinically important blood group antigens: for instance RhD, RhC, Rhc, RhE, Rhe, S, s, Fy^{a}, Fy^{b}, K, k, Kp^{a}, Kp^{b}, Di^{a}, Di^{b}, Jk^{a}, Jk^{b}, and the platelet antigens, Human Platelet Antigen (HPA)-1a and HPA-1b. Important carbohydrate blood groups like ABO, Lewis, P₁/P₂ and others can now also be tested for.

The nucleotide sequence may be a marker, probe, primer or a primer set. The identity/identity may be at least 91, 92, 93, 94, 95, 96, 97, 98 or 99% to the sequence shown in SEQ ID NO:1-6.

In another aspect the invention relates to a method for P¹/P² genotyping analysis comprising the steps of, isolation and purification of DNA or RNA from a samples using techniques well-known for a person skilled in the art, and determining if the DNA or RNA has P¹ or P² genotype by methods using the nucleotide sequence mentioned above. Different techniques may be used as well as different detection nucleotide sequences such as those mentioned above.

The single base-pair difference that discriminate between P¹/P² may be determined by sequencing using specific primers that are directed to SEQ ID NO 1-6 or being one or more of SEQ ID NO:7-13, such as EXAMPLE 1, wherein the primers should be located in a position that enables the possibility to identify the single base pair shift at position 129 (C or T) in SEQ ID NO 1-3. The method can identify a single base-pair difference in the genomic DNA i.e., determine if a person is homozygous for P¹ and having C, or being heterozygous with C/T at position 129, or being homozygous for P² and having T in position 129. If the sample is homo- or heterozygous and has a C/C or C/T present at this position the phenotype will be P₁, and a T/T homozygocity will give a P₂ phenotype.

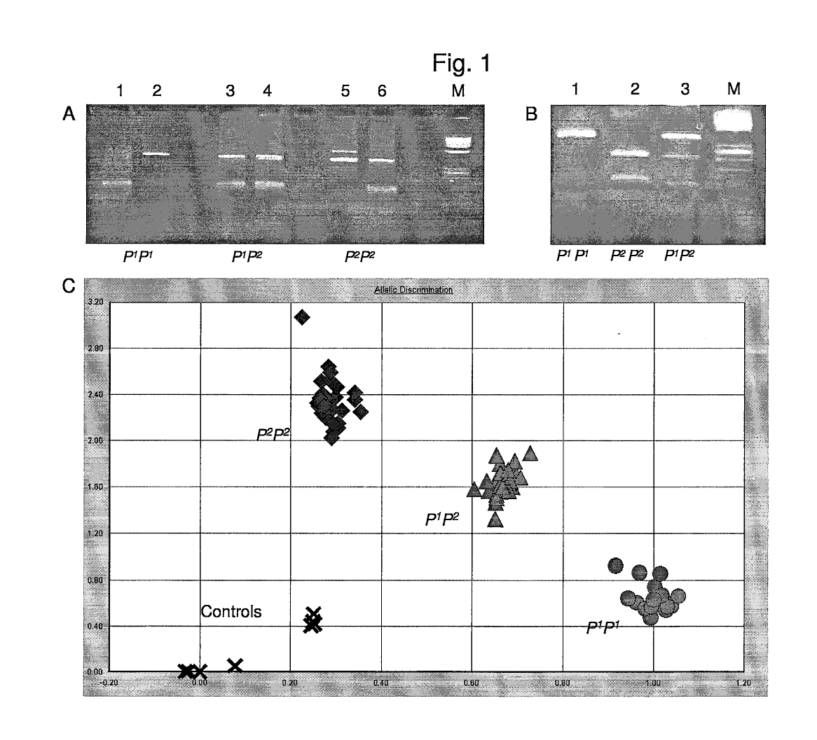

The PCR-Allele Specific Primer (PCR-ASP) technique uses two sets of oligonucleotides comprises one of the two primers being homologous to the P¹/P² alleles at position 129 and being C or T or adjacent to said position. Two separate amplifications each specific for the two different alleles are performed such as EXAMPLE 2. The reactions are detected on an agarose gel and reaction patterns determined, for instance as shown in FIG. 1.

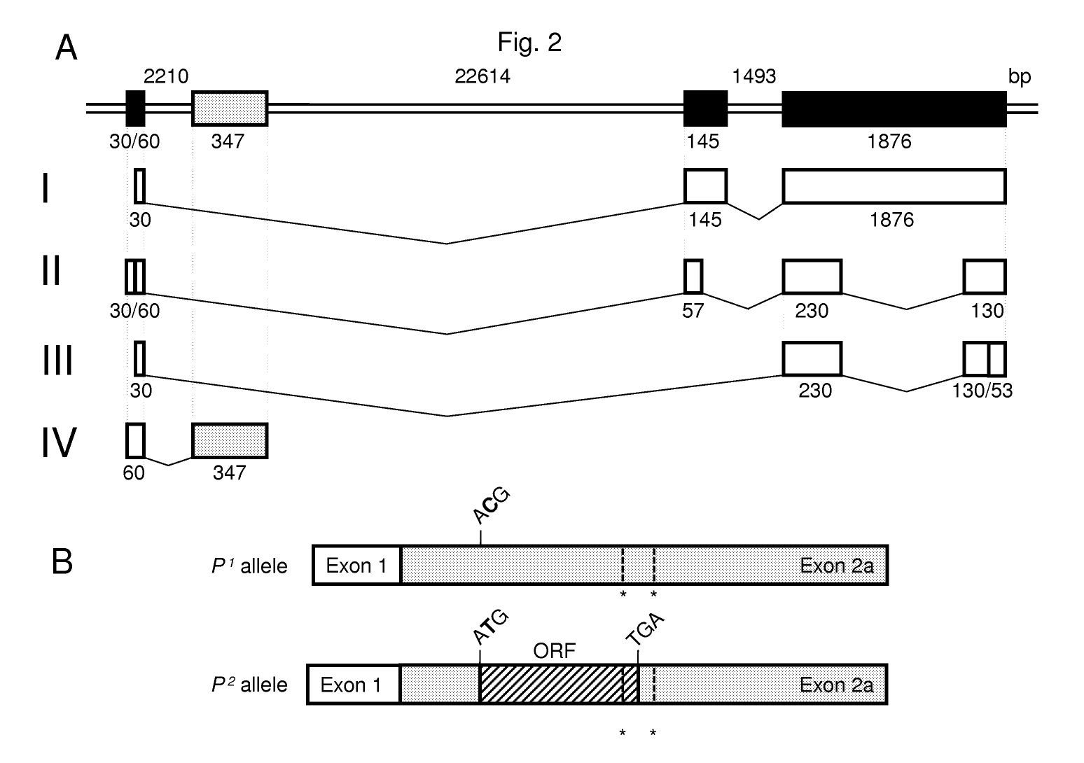

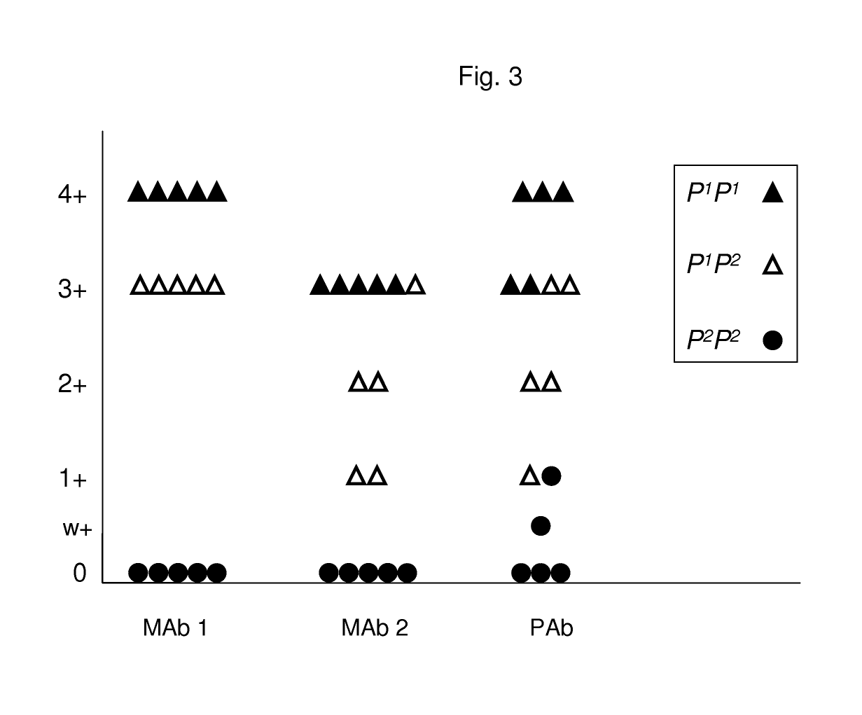

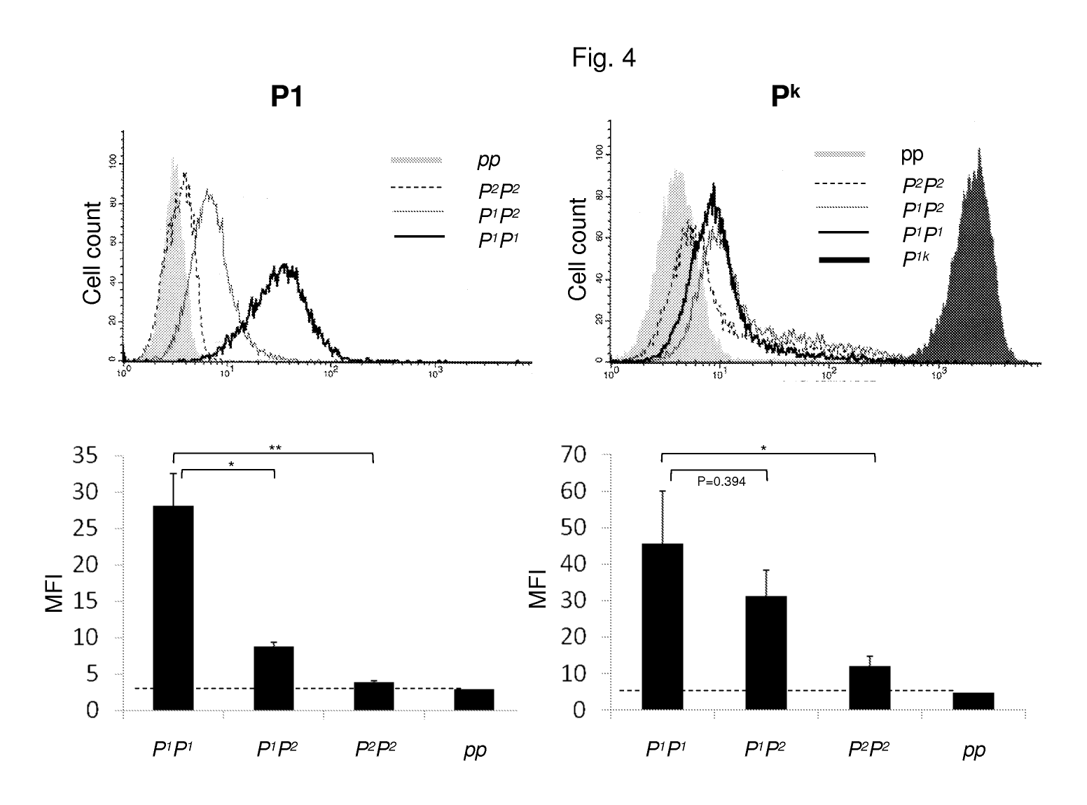

An alternative way would be to first amplify the fragments and then digest them with a suitable restriction enzyme prior to separation on an agarose gel, for instance as shown in FIG. 2.

How to choose methods and technologies that may be used for the development of suitable markers (primers or probes) are well-known for a person skilled in the art. Genomic DNA may be purified using any suitable method such as the Qiagen Blood DNA Isolation Kit (Qiagen Inc. Valencia, Calif., USA). The method(s) can use any good quality DNA harvested by any one of a variety of methods. For the multiplex PCR, the DNA regions containing one or more SNPs of interest may be PCR-amplified in a single reaction well. Note that the concentration of the various reagents may be adjusted to optimize DNA amplification, and is dependent on but is not limited to: the concentration and quality of the genomic DNA, the concentration of the PCR primers or the type of thermal cycler used for the PCR. The amplified PCR products may be separated on an agarose gel and the discrimination between different amplified fragments may determine the P¹/P² phenotypes. Preferably, a control is included to verify that the DNA is pure enough to be amplified by PCR. The control may detect any suitable part of the genome as long as it is not a polymorphic or repetitive sequence.

Other examples of possible methods include but are not limited to allelic discrimination, sequencing, PCR-ASP, PCR-RFLP, pyrosequencing, microarraybead chip array and SNP microarray.

In another aspect the invention relates to an isolated nucleotide sequence showing at least 90% identity to the nucleotide sequence shown in SEQ ID NO 1-3, and having a length at most the same as the nucleotide sequence shown in SEQ ID NO:1-3 wherein position 129 is C or T, such as 95, 96, 97, 98, 99% identity or homology, wherein a person who is homozygous for the allele with C, or heterozygous for the alleles with C and T has the P₁ phenotype and a person homozygous for T has the P₂ phenotype. Identity or homology being defined above. The isolated nucleotide sequence may also be identical to the nucleotide sequence SEQ ID NO 1-6.

In a further aspect the invention relates to a kit comprising primer and/or probe or oligonucleotides having a length of from about 10 to about 30 nucleotides and being homologous to the nucleotide sequences shown in SEQ ID NO:1-6 or showing 90% identity to the nucleotide sequences shown in SEQ ID NO:1-6, such as having a length of about 15-25 nucleotides. Examples of different lengths include 11, 12, 13, 14, 15, 16, 17, 18, 19, 20, 21, 22, 23, 24, 25, 26, 27, 28 or 29 nucleotides. Examples of different homologies includes 95, 96, 97, 98, 99% identity or being identical.

In another aspect the invention relates to a kit comprising a set of oligonucleotide primers comprising a sense and an antisense primer wherein said set of oligonucleotide primers is suitable for amplifying and detecting the P¹/P² genotype as defined above, wherein a person who is homozygous for the allele with C, or heterozygous for the alleles with C and T has the P₁ phenotype and a person homozygous for T has the P₂ phenotype.

The set of primers may have the same technical features as the primer and/or probe defined above. They may be used alone or together with other blood group phenotypes that make multiple analyses possible. The invention represents a novel addition to screening for blood groups and addresses a clear need in the art for novel, rapid, cost-effective and reliable genotyping. This additionally replaces the use of expensive and difficult-to-obtain serological reagents, which can be reserved for use to confirm only the donors identified by the screening process.

Finally, the invention relates to a kit comprising a primer and/or probe or a set of oligonucleotide primers, selected from the group consisting of SEQ ID NO:7-13.

The invention also relates to a method to analyse the expression of the P1 antigen and/or the PK antigen comprising the steps of, determining the expression of the P1 antigen and/or the PK antigen in said sample comprising red blood cells. The determination may be performed by flow cytometry and/or haemaglutination.

The following examples are intended to illustrate, but not to limit, the invention in any manner, shape, or form, either explicitly or implicitly.

### EXAMPLES

### Example 1

### Genotyping by Sequencing

A 545/546 bp fragment including the P¹/P²-specific polymorphism was amplified with the following primers.

| Primer no. | Primer name | Sequence (5′ → 3′) |
|---|---|---|
| 1 | 2145-F | TGAATTAACCGAAAGAAGTAGG |
| 2 | 2648-R | ATGACATAGCAAAATGCAAGCA |

Primer 1=SEQ ID NO:7 and primer 2=SEQ ID NO:8

Amplification mix

| | | |
|---|---|---|
| 2.5 | μl | 10x buffer |
| 0.5 | μl | dNTP (10 mM/nucleotide) |
| 0.8 | μl | 10 mM forward primer |
| 0.8 | μl | 10 mM reverse primer |
| 2.5 | μl | DNA 100 ng/μl |
| 17.5 | μl | H₂O |
| 0.4 | μl | Taq Polymerase 5 U/μl |
| 25 | μl | Total |

PCR programme 1

| | | | |
|---|---|---|---|
| 96° C. | 3 | min | |
| 96° C. | 30 | sec | 10 cycles |
| 62° C. | 30 | sec | |
| 68° C. | 1 | min | |
| 96° C. | 30 | sec | 25 cycles |
| 60° C. | 30 | sec | |
| 68° C. | 1 | min | |
| 72° C. | 1 | min | |

All amplification products were separated by high-voltage electrophoresis on 3% agarose gels (Seakem, FMC Bioproducts, Rockland, Me., USA) stained with ethidium bromide (0.56 mg/l gel, Sigma Chemicals, St. Louis, Mo., USA). Products were purified using the Qiaquick gel extraction kit (Qiagen GmbH, Hilden, Germany), sequenced with the BigDye terminator kit v1.1 (Applied Biosystems) and analysed on a 3130 Avant/Genetic analyser (Applied Biosystems).

An amplification mix was made containing: 1×PCR-buffer I (Applied Biosystems), 2 pmol of each dNTP (Applied Biosystems), 4 pmol forward primer Pk i1 2145F, 4 pmol reverse primer Pk i1 2648R, 100 ng DNA dissolved in H₂O, 0.5 U Taq Gold Polymerase (Applied Biosystems). The amplification reaction was executed in the GeneAmp® PCR System 2700 (Applied Biosystems). PCR conditions: 96° C. 7 min , (94° C. 30 sec, 62° C. 30 sec) for 30 cycles, and 72° C. 20 sec. The same primers were used for sequencing.

The P¹/P²-specific polymorphism is located at position 129 in this fragment.

The short novel exon named 2a was sequenced in 134 samples from individuals with the P₁ and P₂ phenotypes. Three polymorphic sites were identified, in exon 2a at positions 42C>T, 122T>G and 135C>delC (where nucleotide 1 is the first residue in exon 2a). The two polymorphisms at positions 122 and 135 did not correlate well with the P₁/P₂ phenotypes, while the polymorphic site at position 42 in exon 2a did. This corresponds to position 129 in SEQ ID NO: 1-6. All P₂ samples were homozygous for thymidine (T) at this position while the P₁ samples were either homozygous for cytosine or heterozygous. The nucleotide substitution at position 42 in exon 2a from C to T introduces a start codon in the P² allele, which gives rise to a short potential open reading frame (ORF) of 28 amino acids (FIG. 2). One of the other polymorphic sites (122T/G in exon 2a) tentatively changes the last residue from Gly28Trp, thus resulting in two variants of the P²-related ORF. In order to confirm these genetic findings and also investigate how strong the correlation is between genotype and P₁/P₂ phenotypes, more samples were tested with different genotyping methods.

### Example 2

### PCR-ASP

A PCR method was set up using allele-specific primers (ASP). To increase the specificity, mutations from guanosine (G) to adenosine (A) were introduced at position −3 in both reverse primers.

Two reactions, each detecting one of the alleles, were run under the same conditions (shown below). Both reaction mixes contain control primers detecting a non-polymorphic gene.

| Primer no. | Primer name | Sequence (5′ → 3′) |
|---|---|---|
| 3 | F-P1P2 | TTGAATGAATTAACCGAAAGAAGTAGG |
| 4 | P1-Rm | CCAAAGTGCTGGGATTACAGACG |
| 5 | P2-Rm | CCAAAGTGCTGGGATTACAGACA |
| 6 | F-control | GCATGCTGCCATAGGATCATTGC |
| 7 | R-control | GAGCCAGGAGGTGGGTTTGCC |

Primer 3=SEQ ID NO:9, Primer 4=SEQ ID NO:10, primer 5=SEQ ID NO:11, primer 6=SEQ ID NO:14 and primer 7=SEQ ID NO:15

Amplification mix

| | | |
|---|---|---|
| 1.1 | μl | 10x buffer |
| 0.2 | μl | dNTP (10 mM/nucleotide) |
| 0.2 | μl | Glycerol 100% |
| 0.1 | μl | Cresol red 10 mg/ml |
| 0.4 | μl | 10 mM forward primer |
| 0.4 | μl | 10 mM reverse primer |
| 0.08 | μl | 10 mM forward primer control |
| 0.08 | μl | 10 mM reverse primer control |
| 1 | μl | DNA 100 ng/μl |
| 7.35 | μl | H₂O |
| 0.09 | μl | Taq Polymerase 5 U/μl |
| 11 | μl | Total |

PCR programme 2

| | | | |
|---|---|---|---|
| 96° C. | 7 | min | |
| 94° C. | 30 | sec | 30 cycles |
| 62° C. | 30 | sec | |
| 72° C. | 20 | sec | |
| 72° C. | 1 | min | |

201 samples were analyzed, with two primer mixes identifying either the P¹/or P² alleles. Mutations from G to A were introduced in the −3 position in both the P1 and P2 reverse primers to increase the specificity for each allele. Control primers F and R were included shown in the table above.

Amplification mixes with a total volume of 11 μl contains: 1×PCR-buffer (Applied Biosystems, Foster City, Calif., USA). 2 pmol of each dNTP, 4 pmol Pk-P1P2-F, 4 pmol Pk-P1m-R or Pk-P2m-R, 0.8 pmol forward control primer, 0.8 pmol reverse control primer, 100 ng DNA, 0.5 U Taq Gold Polymerase (Applied Biosystems). The amplification reaction was performed in the GeneAmp® PCR System 2700 (Applied Biosystems). Initial denaturation at 96° C. 7 min was followed by 30 cycles (94° C. 30 sec, 62° C. 30 sec, 72° C. 20 sec) to amplify the P¹/P² polymorphism and finally 72° C. for 1 min of elongation. The amplification reaction was detected on agarose gel (3%). The primers used are described above.

| | Polymorphic site | | | |
|---|---|---|---|---|
| Phenotype | C/C | C/T | T/T | Total n |
| P₁ | 55 | 90 | 1 | 146 |
| P₂ | 0 | 0 | 55 | 55 |

A total of 201 random donor blood samples were typed by serology and tested for the P₁ vs. P₂-discriminating SNP by the PCR-ASP-based genotyping method (see table above). Two hundred samples showed full concordance between phenotype and genotype. Only in one case was a discrepancy noted: This sample tested as very weakly positive for P1 by serological routine methods but was found to be homozygous for T at position 42 in exon 2a. A new sample from this donor could not be obtained but flow cytometric semiquantification was performed and confirmed the serological typing although the P1 level was extremely low, the lowest observed in the study (data not shown).

### Example 3

### Genotyping by PCR-RFLP

A 345/346 bp fragment including the P¹/P²-specific polymorphism was amplified with the following primers.

| Primer no. | Primer name | Sequence (5′ → 3′) |
|---|---|---|
| 1 | 2145-F | TGAATTAACCGAAAGAAGTAGG |
| 8 | Ex2a-240-R | CTCGGCTCACTGCACTCTCT |

Primer 1=SEQ ID NO:12 and primer 8=SEQ ID NO:13

Amplification mix

| | | |
|---|---|---|
| 2.5 | μl | 10x buffer |
| 0.5 | μl | dNTP (10 mM/nucleotide) |
| 0.8 | μl | 10 mM forward primer |
| 0.8 | μl | 10 mM reverse primer |
| 2.5 | μl | DNA 100 ng/μl |
| 17.5 | μl | H₂O |
| 0.4 | μl | Taq Polymerase 5 U/μl |
| 25 | μl | Total |

PCR programme 2

| | | | |
|---|---|---|---|
| 96° C. | 7 | min | |
| 94° C. | 30 | sec | 30 cycles |
| 62° C. | 30 | sec | |
| 72° C. | 20 | sec | |
| 72° C. | 1 | min | |

The fragment was amplified and cleaved with the restriction enzyme NlaIII which recognizes and cleaves the sequence CATG. This restriction site is found in the fragment from P² alleles but not in P¹ alleles. Alternative restriction enzymes that could be used are: CviAII, FatI, FaeI, HinIII and Hsp92II.

| NlaIII cleavage | | Cleavage conditions |
|---|---|---|
| PCR product | 10 μl | Incubation in 37° C. |
| 10 x Buffer | 2 μl | for 1 hour |
| Nla III | 2 μl | |
| H₂O | 6 μl | |
| Total | 20 μl | |

Cleaved PCR products were run on a 4% agarose gel.
P¹ alleles gave a 345/346 bp fragment
P² alleles gave two fragments—130 bp and 219/220 bp
P¹/P² heterozygous samples gave three fragments—130 bp, 219/220 bp and 345/346 bp

The P₁ vs. P₂-discriminating SNP was confirmed by this method in 10 random samples phenotyped for P1 antigen expression to show proof of concept for this method.

### Summary: P¹/P² Genotype Screening

Sequencing shown in EXAMPLE 1 as well as different genotyping methods shown in EXAMPLES 2 and 3 were developed and tested based on the above finding; PCR-ASP (EXAMPLE 2, FIG. 1), PCR-RFLP (EXAMPLE 3, FIG. 1) and allelic discrimination (AD) by a SNP genotyping assay (FIG. 1). All assays showed specific and easily interpretable typing patterns (FIG. 1) compared to sequence data and it was concluded that all three methods could be used for screening purposes as outlined below.

### Example 4

### P₁/P₂ Phenotyping

### P1 Strength Correlates to P¹ Zygosity

Samples were phenotyped for the P1 antigen using one or two commercially available anti-P1 reagents according to routine blood banking procedures. The antisera were CE-labelled reagents approved for clinical use on the European market.

For the fifteen-donor cohort described in FIG. 3, samples were investigated with three different antisera: the murine IgM monoclonal reagents ImmuClone® (clone P3NIL100, Immucor, Roedermark, Germany) and Seraclone® Anti-P1 (clone 650 Biotest, Rockaway, N.J., USA) and goat polyclonal anti-P1 (Immucor, Roedermark, Germany). Agglutinates were scored visually and the reaction strength assigned as negative (−) or positive from weak (+) to the strongest (4+) according to current blood bank practice as described the Technical Manual of the American Association of Blood Banks. Cells of known P₁/P₂ phenotypes were used as controls.

Hemagglutination testing was performed with two monoclonal antibodies and one polyclonal goat antiserum, all used in clinical routine practice for P1 phenotyping. With the monoclonal reagents P²P² samples were all negative and the PP/samples were strongly positive while the P¹P² heterozygous samples showed weaker serological reactions than the homozygous samples. The polyclonal antibody gave similar reaction patterns although displayed broader variation, and in two cases P²P² samples actually gave rise to weakly positive reactions, probably signaling that this goat antiserum is not completely specific for P1 antigen (FIG. 3).

### Example 5

### Flow Cytometry

Red blood cells were also typed by flow cytometry to get a more quantitative measure of P1 and P^{k} blood group antigen expression. Cells were washed and diluted in PBS (3% suspension). Incubations were performed in 96-well plates (NUNC™ Apogent, Denmark). 0,07% glutaraldehyde was added in each well and the plate was incubated for 10 min in the dark on a shaking board at room temperature (RT) to fix the RBCs in order to avoid agglutination. After incubation the plate was centrifuged for 1 min in 350×g, the supernatant discarded and the RBCs shook up and diluted in PBS. The primary antibody was added and first incubated for 10 min in the darkness under constant mixing at RT, followed by incubation at 4° C. for 35 minutes. The RBCs were washed twice with PBS before adding the secondary antibody and then incubated for 10 min in the darkness under constant mixing at RT. Primary antibodies for FACS analysis were anti-P^{k} (monoclonal IgM rat antibody CD77, clone38-13, Immunotech, Marseille, France) and anti-P1 (Seraclone® Anti-P1, clone 650, Biotest, Dreieich, Germany). Secondary antibodies were goat F(ab′)² fragments Anti-Rat IgM (mu)-PE (clone IM1625, Immunotech) for anti-P^{k} and PE-conjugated rat-anti-mouse kappa monoclonal (clone X36, Becton Dickinson) for anti-P1. The RBCs were washed twice in PBS after the incubation before they were diluted in PBS. Data were collected with a calibrated FACScan flow cytometer (Becton Dickinson, Calif., USA) and analyzed using Cell Quest software ver 3.1f (Becton Dickinson). PP1P^{k}-negative p phenotype RBCs were used as negative control cells and P₁^{k} RBCs as positive controls.

Independent 2-sample t-test assuming equal variance and 2-tailed distribution was used to determine the significance. Each of the genotype groups (P¹P¹, P¹P² or P²P²) was compared to each other using the XLSTAT 2009(Addinsoft, N.Y., USA) data analyzer. Data were considered statistically significant with respect to the following criteria (*P value<0.05, **P<0.01, ***P<0.001).

The results showed lower expression of P1 antigen on cells with P¹P² genotype than on P¹P¹ cells. P²P² cells had similar expression as cells with the P1 and P^{k} negative p phenotype, thus negative or background levels of fluorescence.

In addition, analysis of P^{k} antigen expression showed that the P^{k} levels on P¹P² heterozygous cells were lower than on P¹P¹ homozygous cells, although the difference was not significant. The P²P² homozygous cells displayed more P^{k} expression than cells with p phenotype but significantly lower than P¹P¹ cells (FIG. 4).

1. A method to discriminate between the P¹ and P² alleles by the use of at least one nucleotide sequence being homologous or complementary to part of the nucleotide sequences as shown in SEQ ID NO: 1-6 or a nucleotide sequence showing at least 90% identity to any of SEQ ID NO:1-6 and wherein the difference between the alleles is a C or T in position 129 as shown in SEQ ID NO:1, wherein a person who is homozygous for the allele with C, or heterozygous for the alleles with C and T has the P₁ phenotype and a person homozygous for T has the P₂ phenotype.

2. The method according to claim 1, wherein said at least one nucleotides sequence is a marker, probe, primer or primer set.

3. The method according to any of claims 1-2, wherein the nucleotide sequence shows at least 95% identity to the sequences shown in SEQ ID NO:1-6.

4. The method according to any of claims 1-3, wherein the nucleotide sequence shows at least 97% identity to the sequences shown in SEQ ID NO:1-6.

5. The method according to any of claims 1-4, comprising the steps of;
a. isolation and purification of DNA from in a sample and
b. determining if the DNA has P¹ or P² genotype by the use of the nucleotide sequence according to claim 1.

6. The method according to claims 1-5, wherein said method is selected from the group consisting of sequencing, PCR-ASP, PCR-RFLP, allelic discrimination, pyrosequencing, microarray or variations thereof.

7. The method according to any of claims 1-6, wherein the ability to discriminate between the P¹ and P² alleles predicts the expression level of the P1 antigen and/or the Pk antigen by zygosity analysis where homozygosity for P1 predicts high antigen levels and heterozygosity for P1 and P2 predicts low levels.

8. An isolated nucleotide sequence showing at least 90% identity to the nucleotide sequence shown in SEQ ID NO 1-3 and having a length being at most the same as the nucleotide sequences shown in SEQ ID NO:1-3 comprising the P¹/P² alleles, and wherein the difference between the alleles is a C or Tin position 129 as shown in SEQ ID NO:1, and wherein a person who is homozygous for the allele with C, or heterozygous for the alleles with C and T has the P₁ phenotype and a person homozygous for T has the P₂ phenotype.

9. A kit comprising a set of oligonucleotide primers being homologous or complementary to the nucleotide sequence shown in SEQ ID NO:1-6 and wherein said set of oligonucleotide primers is suitable for amplifying and/or detecting the P¹/P² genotype and wherein the difference between the alleles is a C or T in position 129 as shown in SEQ ID NO:1, wherein a person who is homozygous for the allele with C, or heterozygous for the alleles with C and T has the P₁ phenotype and a person homozygous for T has the P₂ phenotype.

10. A kit according to claim 9, wherein the set of oligonucleotide are selected from the group consisting of SEQ ID NO:7-13.


SEQUENCE LISTING (15 sequences)

SEQ ID NO: 1 - DNA, 546 bp, Homo sapiens
tgaattaaccgaaagaagtaggaaaaacaattcctgtttgcatacttgaagacaactattgttaacatttgcctttttttttgccagacctttaaaaacacgtttttccagccgggcgaggtggctcacgcctgtaatcccagcactttgggaggctgaggcgggtggatcacttgaagtcaggagttcgagaccagcctggccaacttggtgaaaccccccatctccactaaaaatacaataaattaactgggcgtggtggcacgtgcctgcaatcccagctactcaggaggctgaggcaggagaatcatttgaacctgggaggcagagagtgcagtgagccgagatggcgccactgcactccagcctgagcaacagagcgagactccgtctccaaaaataaataaataaataataaaataacacgtttttctgtatttgcatatcttgtctatataaaatatttttccattagtataatttcacgagcattcctcatcgcagacattttttatatttttcaatgcttgcattttgctatgtcat

SEQ ID NO: 2 - DNA, 545 bp, Homo sapiens
tgaattaaccgaaagaagtaggaaaaacaattcctgtttgcatacttgaagacaactattgttaacatttgcctttttttttgccagacctttaaaaacacgtttttccagccgggcgaggtggctcatgcctgtaatcccagcactttgggaggctgaggcgggtggatcacttgaagtcaggagttcgagaccagcctggccaacttggtgaaacccccatctccactaaaaatacaataaattaactgggcgtggtggcacgtgcctgcaatcccagctactcaggaggctgaggcaggagaatcatttgaacctgggaggcagagagtgcagtgagccgagatggcgccactgcactccagcctgagcaacagagcgagactccgtctccaaaaataaataaataaataataaaataacacgtttttctgtatttgcatatcttgtctatataaaatatttttccattagtataatttcacgagcattcctcatcgcagacattttttatatttttcaatgcttgcattttgctatgtcat

SEQ ID NO: 3 - DNA, 545 bp, Homo sapiens
tgaattaaccgaaagaagtaggaaaaacaattcctgtttgcatacttgaagacaactattgttaacatttgcctttttttttgccagacctttaaaaacacgtttttccagccgggcgaggtggctcatgcctgtaatcccagcactttgggaggctgaggcgggtggatcacttgaagtcaggagttcgagaccagcctggccaactgggtgaaacccccatctccactaaaaatacaataaattaactgggcgtggtggcacgtgcctgcaatcccagctactcaggaggctgaggcaggagaatcatttgaacctgggaggcagagagtgcagtgagccgagatggcgccactgcactccagcctgagcaacagagcgagactccgtctccaaaaataaataaataaataataaaataacacgtttttctgtatttgcatatcttgtctatataaaatatttttccattagtataatttcacgagcattcctcatcgcagacattttttatatttttcaatgcttgcattttgctatgtcat

SEQ ID NO: 4 - DNA, 421 bp, Homo sapiens
ccggccgccgcgccgcccgcccgccggggccccgctgtccgccgcccgccgccgctggagctagagacctttaaaaacacgtttttccagccgggcgaggtggctcacgcctgtaatcccagcactttgggaggctgaggcgggtggatcacttgaagtcaggagttcgagaccagcctggccaacttggtgaaaccccccatctccactaaaaatacaataaattaactgggcgtggtggcacgtgcctgcaatcccagctactcaggaggctgaggcaggagaatcatttgaacctgggaggcagagagtgcagtgagccgagatggcgccactgcactccagcctgagcaacagagcgagactccgtctccaaaaataaataaataaataataaaataacacgtcaaaaaaaaaaaaa

SEQ ID NO: 5 - DNA, 420 bp, Homo sapiens
ccggccgccgcgccgcccgcccgccggggccccgctgtccgccgcccgccgccgctggagctagagacctttaaaaacacgtttttccagccgggcgaggtggctcatgcctgtaatcccagcactttgggaggctgaggcgggtggatcacttgaagtcaggagttcgagaccagcctggccaacttggtgaaacccccatctccactaaaaatacaataaattaactgggcgtggtggcacgtgcctgcaatcccagctactcaggaggctgaggcaggagaatcatttgaacctgggaggcagagagtgcagtgagccgagatggcgccactgcactccagcctgagcaacagagcgagactccgtctccaaaaataaataaataaataataaaataacacgtcaaaaaaaaaaaaa

SEQ ID NO: 6 - DNA, 395 bp, Homo sapiens
ccggccgccgcgccgcccgcccgccgccgctggagctagagacctttaaaaacacgtttttccagccgggcgaggtggctcatgcctgtaatcccagcactttgggaggctgaggcgggtggatcacttgaagtcaggagttcgagaccagcctggccaactgggtgaaacccccatctccactaaaaatacaataaattaactgggcgtggtggcacgtgcctgcaatcccagctactcaggaggctgaggcaggagaatcatttgaacctgggaggcagagagtgcagtgagccgagatggcgccactgcactccagcctgagcaacagagcgagactccgtctccaaaaataaataaataaataataaaataacacgtcaaaaaaaaaaaaa

SEQ ID NO: 7 - DNA, 22 bp, Homo sapiens
tgaattaaccgaaagaagtagg

SEQ ID NO: 8 - DNA, 22 bp, Homo sapiens
atgacatagcaaaatgcaagca

SEQ ID NO: 9 - DNA, 27 bp, Homo sapiens
ttgaatgaattaaccgaaagaagtagg

SEQ ID NO: 10 - DNA, 23 bp, Homo sapiens
ccaaagtgctgggattacagacg

SEQ ID NO: 11 - DNA, 23 bp, Homo sapiens
ccaaagtgctgggattacagaca

SEQ ID NO: 12 - DNA, 22 bp, Homo sapiens
tgaattaaccgaaagaagtagg

SEQ ID NO: 13 - DNA, 20 bp, Homo sapiens
ctcggctcactgcactctct

SEQ ID NO: 14 - DNA, 23 bp, Homo sapiens
gcatgctgccataggatcattgc

SEQ ID NO: 15 - DNA, 21 bp, Homo sapiens
gagccaggaggtgggtttgcc